(19) United States
(12) Patent Application Publication
(10) Pub. No.: US 2016/0135798 A1
(43) Pub. Date: May 19, 2016

(54) SURGICAL CAPTURE DEVICE AND METHODS OF USE

(71) Applicants: Kirstie Macleod, North Somerset (GB); James Howard, North Somerset (GB)

(72) Inventors: Kirstie Macleod, North Somerset (GB); James Howard, North Somerset (GB)

(73) Assignee: ESPINER MEDICAL LTD., NORTH SOMERSET (GB)

(21) Appl. No.: 14/944,186

(22) Filed: Nov. 17, 2015

Related U.S. Application Data
(60) Provisional application No. 62/080,794, filed on Nov. 17, 2014.
(60) Provisional application No. 62/080,799, filed on Nov. 17, 2014.

(51) Int. Cl. A61B 17/00; A61B 17/221

(52) U.S. Cl. CPC A61B 17/00234; A61B 17/221; A61B 2017/00287; A61B 17/3205

(57) ABSTRACT

A surgical capture device is described that is configured to sequester tissue from a surgical cavity while the tissue is morcellated, or otherwise reduced in size. The capture device includes a side conduit that provides access to the interior of the device so that the tissue can be visualized during the morcellation process. Once the tissue has been removed, the capture device, including the conduit, can be removed from the subject thereby minimizing the risk that any of the morcellated tissue is distributed within the subject. The device is well-suited for procedures such as laparoscopic hysterectomy with power morcellation removal of the uterus and or uterine fibroids.

### CROSS-REFERENCE TO RELATED APPLICATIONS

This application claims the benefit of, and priority to, U.S. Provisional Application Ser. No. 62/080,794, filed January Nov. 17, 2014, and U.S. Provisional Application Ser. No. 62/080,799, filed Nov. 17, 2014, the content of each of which is incorporated by reference herein in its entirety.

### FIELD OF THE INVENTION

The invention relates to surgical devices that are used to recover tissues from an operative field.

### BACKGROUND

Laparoscopic techniques allow a variety of surgical techniques to be completed without making a large incision in the patient. This method of minimally-invasive surgery results in reduced recovery times, reduced medical costs, and reduced morbidity associated with the procedure itself, e.g., due to infection. Millions of laparoscopic surgeries are performed in the United States each year, including cholecystectomies and hysterectomies.

Because laparoscopic surgery is typically done through trocars only about a few centimeters in diameter, additional steps must be taken to morcellate (break into smaller pieces) tissues that are to be removed. For smaller tissue collections, such as a small gallbladder, the tissue may be morcellated by merely cutting with a laparoscopic cutter and aspirating the cut tissue. For larger tissue removal, such as a uterus, a surgical tool, known as a power morcellator, is typically used to morcellate and remove the tissue through a trocar of between 10 and 20 mm. Morcellators are commercially-available from manufactures such as Karl Storz (Tuttlingen, Germany) and Olympus (Tokyo, Japan). Typically the tissue to be morcellated is visualized through a laparoscope that has been inserted through an adjacent trocar. This “side-view” technique provides a greater field of vision so that a surgeon can verify that the correct tissue is being morcellated. The “side-view” technique is also less technically-challenging because the laparoscope can be held out of the way of the morcellator. Furthermore, placing the laparoscope and the morcellator though the same trocar, e.g., using a multi-port trocar, requires more expensive, precision-manufactured, trocars.

In some embodiments, the tissue to morcellated is visualized via one laparoscopic port using a laparoscope while the tissue is morcellated through a separate laparoscopic port. In other embodiments, the tissue is visualized, morcellated, and removed through a single port. If a single port is used, a single-site access device, such as GELPOINT™ (Applied Medical, Rancho Santa Margarita, Calif.) or SILS™ port (Covidien, Mansfield, Mass.) is typically used to allow all of the needed instruments to be used through the same port. While single-site techniques conveniently require only one incision, the single-site access devices are reasonably expensive ($300-$800), and increase the cost of a procedure.

Recently, some hospitals and regulatory agencies have expressed concern that power morcellation may lead to the unintended distribution of cancerous cells inside the body, which could result in malignancies. In particular, the U.S. Food and Drug Administration issued a safety communication in 2014 discouraging the use of laparoscopic power morcellation for removal of uterine or uterine fibroid tissues. See “Laparoscopic Uterine Power Morcellation in Hysterectomy and Myomectomy: FDA Safety Communication,” United States Food and Drug Administration, Apr. 17, 2014, incorporated by reference herein in its entirety.

In view of the many benefits of laparoscopic surgery, there is a need for improved methods of sequestering tissue prior to morcellation to reduce the potential for the spread of cancerous cells, or other unintended consequences of distributing morcellated tissues.

### SUMMARY OF THE INVENTION

The invention is a surgical capture device configured for recovering tissue during minimally invasive surgery, e.g., using laparoscopic techniques. In one embodiment, the surgical capture device comprises a pouch having a main opening for placing tissue within the pouch. The surgical capture device additionally includes a side conduit with a second opening that provides access to the interior of the pouch from another port. Using the second port, is possible to visualize a tissue placed within the pouch, e.g., with a laparoscope, or to address the interior of the pouch with other instruments. Using the surgical capture device, it is possible to morcellate, or otherwise reduce the bulk of, tissue that has been dissected during the surgery, while minimizing the risk that the dissected tissue will be inadvertently distributed in the surgical field. In some embodiments, the surgical capture device will include one or more closure mechanisms to reduce the size of the first or second openings.

In one embodiment, generally, the surgical capture device of the invention will be used to isolate tissues that are to be removed during minimally-invasive surgery using two or more trocars (or ports or trocar sites). The surgical capture device is disposed in a cavity of the subject, whereupon the tissue to be removed is placed inside the device. Next the main opening is extracted through a first trocar (or port or trocar site), and the conduit, including the second opening, is extracted through the second trocar (or port or trocar site)A laparoscope is then passed through the second trocar to visualize the tissue inside the device. Under visualization, the tissue inside the device can be morcellated with a cutter or power morcellator so that the tissue can be removed from the body. Once the tissue has been removed, the entire surgical capture device can be removed from the subject and discarded.

In another embodiment, the surgical capture device comprises a pouch having an opening for placing tissue within the pouch. The surgical capture device additionally includes a plurality of guides adjacent to the opening for guiding additional instruments to be used through the same opening. The guides allow a surgeon to visualize a tissue placed within the pouch, e.g., with a laparoscope inserted through a guide, while addressing the interior of the pouch with other instruments. Using the surgical capture device, it is possible to morcellate, or otherwise reduce the bulk of, tissue that has been dissected during the surgery, while minimizing the risk that the dissected tissue will be inadvertently distributed in the surgical field. In some embodiments, the surgical capture device will include a closure mechanism to reduce the size of the opening.

In general, this embodiment of the surgical capture device of the invention will be used to isolate tissues that are to be removed during minimally-invasive surgery using a single trocar (or port or trocar site). The surgical capture device is disposed in a cavity of the subject, whereupon the tissue to be removed is placed inside the device. Next the opening is extracted through the trocar or port or trocar site, and the opening pushed open to provide access to the guides. A laparoscope is then passed through a guide to visualize the tissue inside the device. Under visualization, the tissue inside the device can be morcellated with a cutter or power morcellator by placing that tool in a separate guide or by inserting the tool through the opening, not using a guide. Once the tissue has been removed, the entire surgical capture device can be removed from the subject and discarded.

In some embodiments, the surgical capture device is constructed from nylon fabric, such as rip-stop nylon. The device may be lined with an impervious barrier, such as polyurethane or polyethylene, or the fabric from which the device is constructed may be coated with an impervious barrier, e.g., comprising polyethylene or polyurethane. In some embodiments, the barrier may be semi-permeable in that very small molecules (e.g., water) can pass through the barrier while larger molecules (e.g., proteins) and cells cannot pass through the barrier. In some embodiments, the seams of the device are sewn together.

### BRIEF DESCRIPTION OF THE DRAWINGS

FIG. 1A shows a portion of the uterus and cervix removed during laparoscopic hysterectomy.

FIG. 1B shows a portion of the uterus and cervix removed during laparoscopic hysterectomy.

FIG. 2 shows a possible placement of multiple trocars for laparoscopic hysterectomy.

FIG. 3A shows one embodiment of a surgical capture device comprising a single conduit for communication between a second laparoscopic port and an interior of the surgical capture device wherein tissues will be morcellated.

FIG. 3B shows another embodiment of a surgical capture device comprising dual conduits for communication between second and third laparoscopic ports and an interior of the surgical capture device wherein tissues will be morcellated.

FIG. 4A depicts delivering a tissue to an interior of the surgical capture device after the device has been deployed to a cavity of a subject.

FIG. 4B depicts delivering a tissue to an interior of the surgical capture device after the device has been deployed to a cavity of a subject.

FIG. 4C depicts reducing the size of a first opening of the surgical capture device by manipulating a first closure mechanism.

FIG. 4D depicts extracting the first opening through a first port by pulling on the closure mechanism, which is a pull-cord.

FIG. 4E shows the first opening re-opened to allow passage of one or more laparoscopic instruments.

FIG. 4F depicts extracting the conduit through a second port.

FIG. 4G depicts the surgical capture device with the first opening through a first port and the second opening through the second port. The second opening provides access to the interior of the pouch while maintaining an impervious (or semi-permeable) barrier around the tissue that is to be morcellated.

FIG. 4H depicts insertion of a laparoscope through the second opening, thereby allowing visualization of the tissue to be removed.

FIG. 4I depicts removal of the tissue with a power morcellator with laparoscopic visualization through a second port.

FIG. 4J depicts retracting the conduit into the interior of the pouch by manipulating a retraction mechanism.

FIG. 4K depicts removal of the surgical capture device after the tissue has been removed.

FIG. 5 shows a possible placement of a single port for laparoscopic hysterectomy.

FIG. 6A shows a side perspective view of another embodiment of a surgical capture device comprising guides;

FIG. 6B shows a top view of a surgical capture device comprising guides;

FIG. 7A depicts delivering a tissue to an interior of the surgical capture device after the device has been deployed to a cavity of a subject;

FIG. 7B depicts delivering a tissue to an interior of the surgical capture device after the device has been deployed to a cavity of a subject;

FIG. 7C depicts reducing the size of an opening of the surgical capture device by manipulating a closure mechanism;

FIG. 7D depicts extracting the opening through a port by pulling on the closure mechanism, which is a pull-cord;

FIG. 7E shows the opening after it is re-opened to allow passage of one or more laparoscopic instruments;

FIG. 7F depicts a surgical capture device with two guides, and having a power morcellator, laparoscope, and a source of gas passing through an opening to address tissue that is to be removed.

### DETAILED DESCRIPTION

A surgical capture device is described that is configured to sequester tissue from a surgical cavity while the tissue is morcellated, or otherwise reduced in size. One embodiment of a capture device consistent with the present disclosure includes a side conduit that provides access to the interior of the device so that the tissue can be visualized during the morcellation process. Once the tissue has been removed from the device, the capture device, including the conduit, can be removed from the subject, thereby minimizing the risk that any of the morcellated tissue is distributed within the subject. The device is well-suited for procedures such as laparoscopic hysterectomy using power morcellation to remove the uterus and/or uterine fibroids.

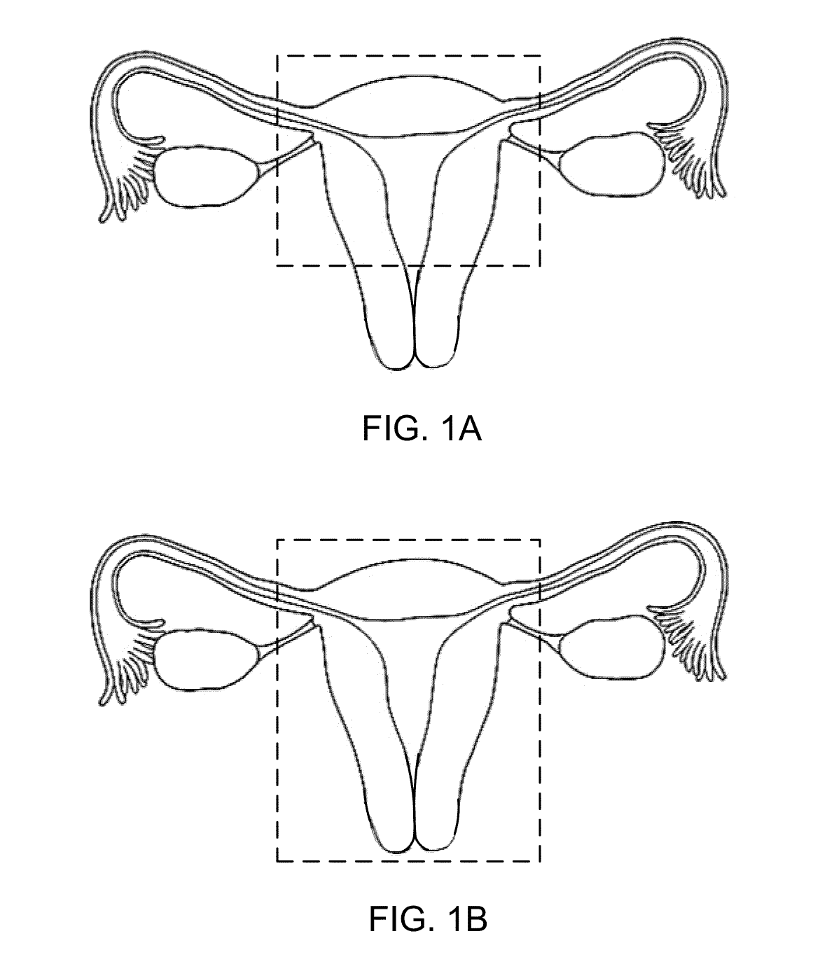

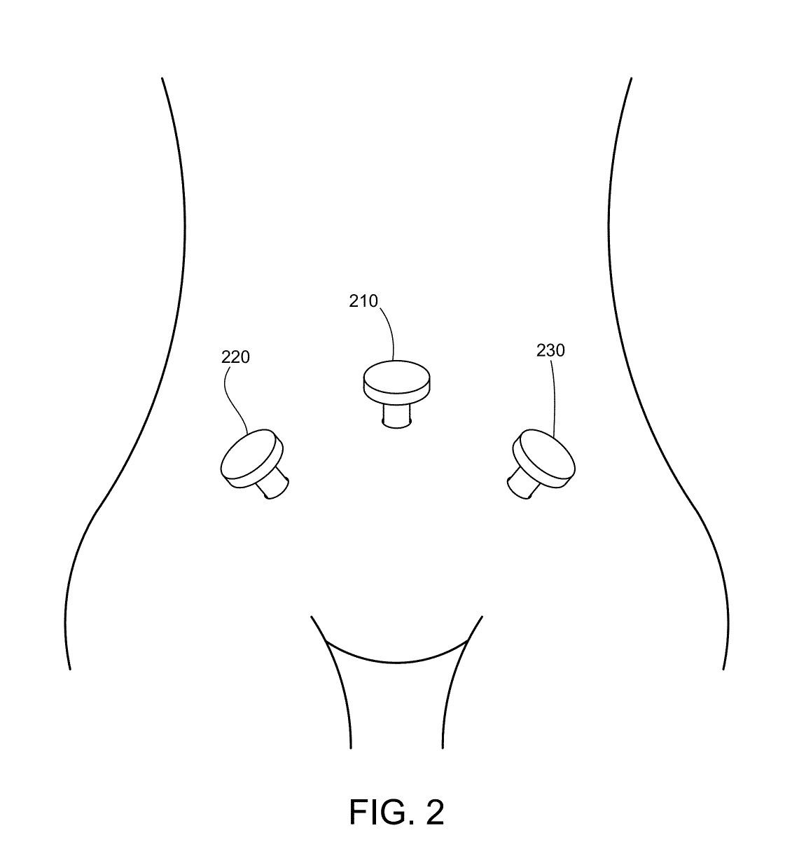

Laparoscopic hysterectomy involves the removal of the uterus through one or more small incisions in the belly. In some laparoscopic hysterectomy procedures, the body of the uterus is removed, but the cervix is left intact, as shown in FIG. 1A. Typically, the ovaries are left intact unless the patient has a family history of ovarian cancer or other risk factors for ovarian disease. This procedure typically involves placement of trocars in the umbilicus (belly button) and in the lower left and right quadrants of the pelvis, as shown in FIG. 2. As will be described in greater detail herein, in other situations, the procedure may be completed through a single incision, using a single trocar or port, as shown in FIG. 5.

Initially the belly is insufflated with carbon dioxide and the uterus is visualized through the umbilicus trocar and surveyed for excessive adhesions or other defects that contraindicate laparoscopic hysterectomy. Once visualized, the uterus can be manipulated with a uterine manipulator while a cutting instrument (laparoscopic scalpel, cautery, laparoscopic scissors, etc.) and forceps are used to dissect the tissues and coagulate and cut the utero-ovarian ligaments. Once the ligaments are severed, dissection proceeds, and the uterine arteries and veins are identified. The blood vessels are sealed and severed, and the body of the uterus is cut from the cervix resulting in an intact uterus. While a healthy uterus is typically about the size of an avocado, some postpartum uteruses and diseased uteruses are much larger, e.g., the size of a grapefruit or larger. In some laparoscopic hysterectomy procedures, the uterus and the cervix are cut from the pelvic floor and removed, as shown in FIG. 1B.

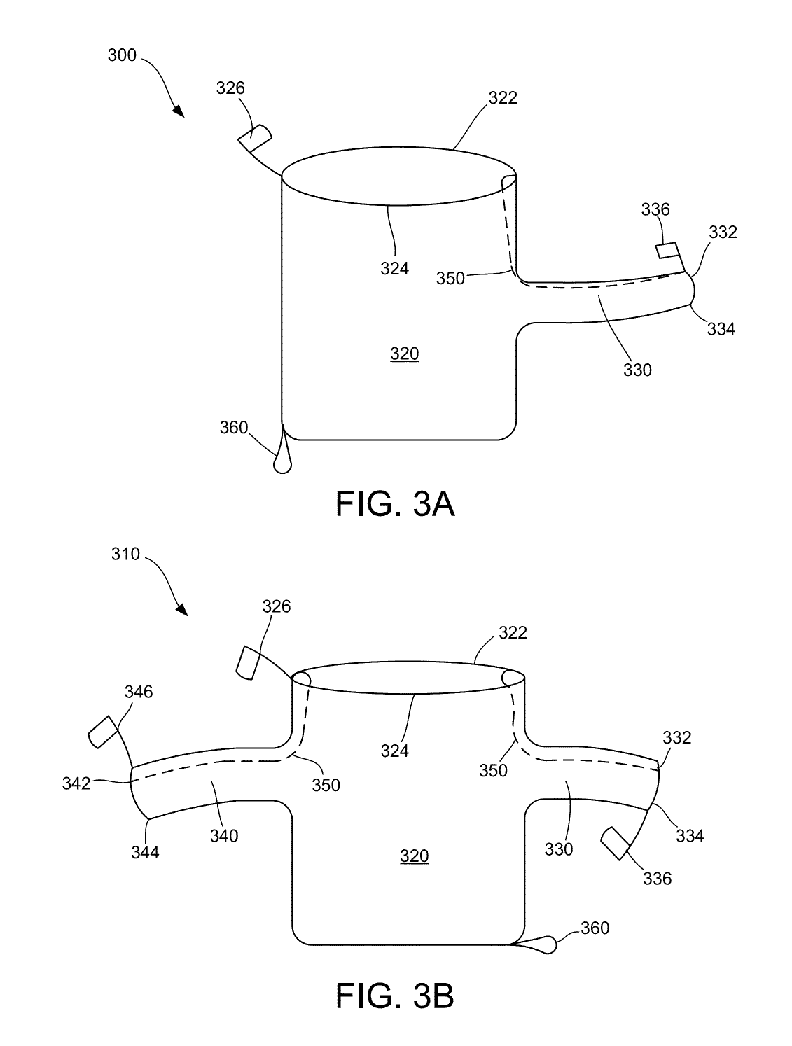

FIGS. 3A and 3B show two embodiments of a surgical capture device of the invention. The surgical capture devices 300 and 310 include a pouch 320 formed by joining sheet material such as fabric or plastic. Device 300 is a surgical capture device including a single conduit 330. Device 310 is a surgical device including two conduits, 330 and 340. Each device 300 and 310 has a main opening 322 that provides a large passageway to the interior of the pouch 320. A closure mechanism 324 surrounds the main opening 322 and allows a user to reversibly reduce the size of the main opening 322. In some embodiments, the closure mechanism 324 is coupled to a pull-cord 326 that makes it easy to actuate the closure mechanism 324 with a tool, such as laparoscopic forceps.

The conduit 330 provides a passage from a secondary opening 332 to the interior of the pouch 320. In some embodiments, a closure mechanism 334 surrounds the secondary opening 332, and allows a user to reversibly reduce the size of the secondary opening 332. In some embodiments, the closure mechanism 334 is coupled to a pull-cord 336 that makes it easy to actuate the closure mechanism 334 with a tool, such as laparoscopic forceps. In the embodiment shown in FIG. 3A, the device 300 additionally comprises a retraction mechanism 350 that allows the conduit 330 to be retracted into the interior of the pouch 320. The retraction mechanism 350 is typically used to make sure that the conduit has been completely contained within the pouch 320 as the device 300 is removed from the subject after morcellation has been completed. Additionally, in many embodiments, the device 300 includes a pull-tab 360 on the exterior of the pouch 320 opposite the main opening 322. The pull-tab 360 provides a point to pull the back of the device to either cause it to move within the surgical cavity, or to help stretch out the device 300, for example, for final extraction from the subject.

In embodiments having more than one conduit 330, such as device 310 depicted in FIG. 3B, each additional conduit 340 may also comprise a closure mechanism 344 surrounding the tertiary opening 342 that allows a user to reversibly reduce the size of the tertiary opening 342. In some embodiments, the closure mechanism 344 is coupled to a pull-cord 346 that makes it easy to actuate the closure mechanism 344 with a tool, such as laparoscopic forceps. The additional conduits 340 can provide access to the interior of the pouch 320 for other surgical tools, such as insufflation tubes, aspiration tubes, cautery tools, etc. Each additional conduit 340 may also be retracted with a retraction mechanism 350 that allows the additional conduit 340 to be retracted into the interior of the pouch 320, as shown in FIG. 3B. There is no limit to the number of conduits that can be included with the invention. In some embodiments, the device of the invention will include at least two conduits, e.g., three conduits, e.g., four conduits, e.g., five conduits, e.g., six conduits, e.g., seven conduits, e.g., eight conduits.

In some embodiments, the surgical capture device 300, 310, is made from fabric, such as nylon or polyester. In some embodiments, the fabric is reinforced with additional threads, such as rip-stop nylon fabric. The reinforcement may run in multiple directions in the fabric to produce, e.g., a cross-hatch or diamond pattern. In some embodiments, the reinforcement may include polymer member or metal wires. The reinforcement prevents instruments from perforating the fabric, and in the event that the fabric is perforated, the reinforcements will prevent the fabric from tearing further. In addition to the structural fabric, the surgical capture device 300, 310 may additionally comprise a lining or a coating to make the device 300, 310 impervious to fluids, e.g., blood or other body fluids. In some embodiments, the lining will comprise polyethylene, polypropylene, or polyurethane. The same materials may alternatively, or in addition, be used to coat the fabric to produce a surgical capture device 300, 310 that is impervious to fluids. In some embodiments, the lining may create a semi-permeable barrier in that small molecules, such as water and some gasses can pass through the barrier while larger molecules, such as proteins, nucleic acids, and lipids cannot pass through the barrier.

In an embodiment, the surgical capture device 300, 310 is constructed from a fabric, e.g., rip-stock nylon, and the seams are joined with thread, e.g., using sewing. In alternative embodiments, the seams may be joined with mechanical fasteners or the seams may be joined with adhesive or other joining material such as fabric tape.

The size of the devices 300, 310, of the invention can vary substantially depending upon the needs of the surgeon and the size of the tissue to be removed. For example, the devices 300 and 310 shown in FIGS. 3A and 3B may comprise at least about 100 cm² of fabric, e.g., at least about 150 cm², e.g., at least about 200 cm², e.g., at least about 250 cm², e.g., at least about 300 cm², e.g., at least about 400 cm², e.g., at least about 500 cm², e.g., at least about 1000 cm² of fabric. The insufflated volume of the pouch 320 may be at least about least 1000 cm³, e.g., at least about 1500 cm³, e.g., at least about 2000 cm³, e.g., at least about 2500 cm³, e.g., at least about 3000 cm³, e.g., at least about 3500 cm³, e.g., at least about 4000 cm³. The length of the conduit 330, 340 may be at least about 20 cm in length as determined by measuring from the secondary opening 332 to a corner formed with the outer wall of the pouch 320. The conduit may be longer, however, e.g., at least about 25 cm, e.g., at least about 30 cm, e.g., at least about 35 cm, e.g., at least about 40 cm, e.g., at least about 50 cm. The average circumference of the conduit may be 5 cm or greater, e.g., 10 cm or greater, e.g., 15 cm or greater, e.g., 20 cm or greater.

The main opening 322 may be at least 30 cm in circumference, to allow the tissue to be morcellated to be easily placed within the pouch using laparoscopic tools, such as laparoscopic forceps. In alternative embodiments, the main opening 322 may be greater than 40 cm in circumference, e.g., greater than 50 cm in circumference, e.g., greater than 60 cm in circumference. The secondary opening 332 and the tertiary opening 342 may have a circumference of 5 cm or greater, e.g., 10 cm or greater, e.g., 15 cm or greater, e.g., 20 cm or greater.

A variety of closure mechanisms 324, 334, 344 can be used to reversibly reduce the size of the openings 322, 332, 342. In some embodiments, the closure mechanism is a pull cord, which comprises a single element such as a nylon, polypropylene, or polyvinylidene fluoride filament. As the pull cord is pulled, the openings 322, 332, 342 are reduced in circumference, allowing a tissue to be secured within the device 300, 310. In some embodiments, the pull cord comprises multiple elements, such as multiple nylon filaments. In some embodiments, the main opening 322 may be reinforced with a polymer layer to improve the stiffness of the opening, as opposed to fabric alone, to facilitate opening the device after it has been delivered to the surgical cavity. Embodiments with multiple filaments have a decreased risk of breakage when the pull-cord is pulled, and the additional resilience provided by the extra elements results in a device 300, 310 that is essentially self-opening. The self-opening feature is especially useful because the device is immediately ready to have the tissue deposited after it has been placed through the trocar. In other words, the surgeon does not have to spend additional time opening the opening 322, 332, 342 before the device 300, 310 can be used. Other closure mechanisms 324, 334, 344 may include pull strings, zippers, snaps, or flange-in-channel closures, i.e., ZIP-LOC™-type closure mechanisms. The closure mechanism may have a handle, tab, or flag to make it easier to interact with the closure mechanism during a procedure. The handle, tab or flag may be color-coded or shape-coded to assist in identifying specific closure mechanisms during a procedure. In some embodiments the closure mechanisms will be elongated, such as an elongated pull-cord to provide easier access to the closure mechanism outside of the patient. For example, in some embodiments, a pull cord may be 30 cm in length beyond the length required for the main opening 322 to be completely opened. Other embodiments may include a longer pull cord, e.g., at least 40 cm in length, e.g., at least 50 cm in length.

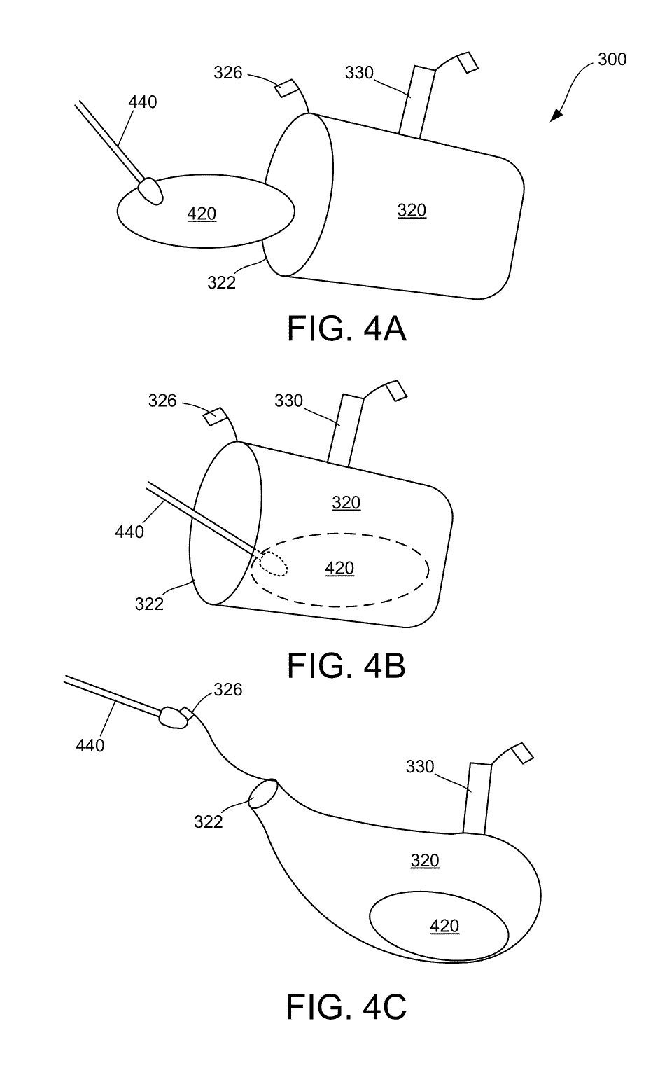

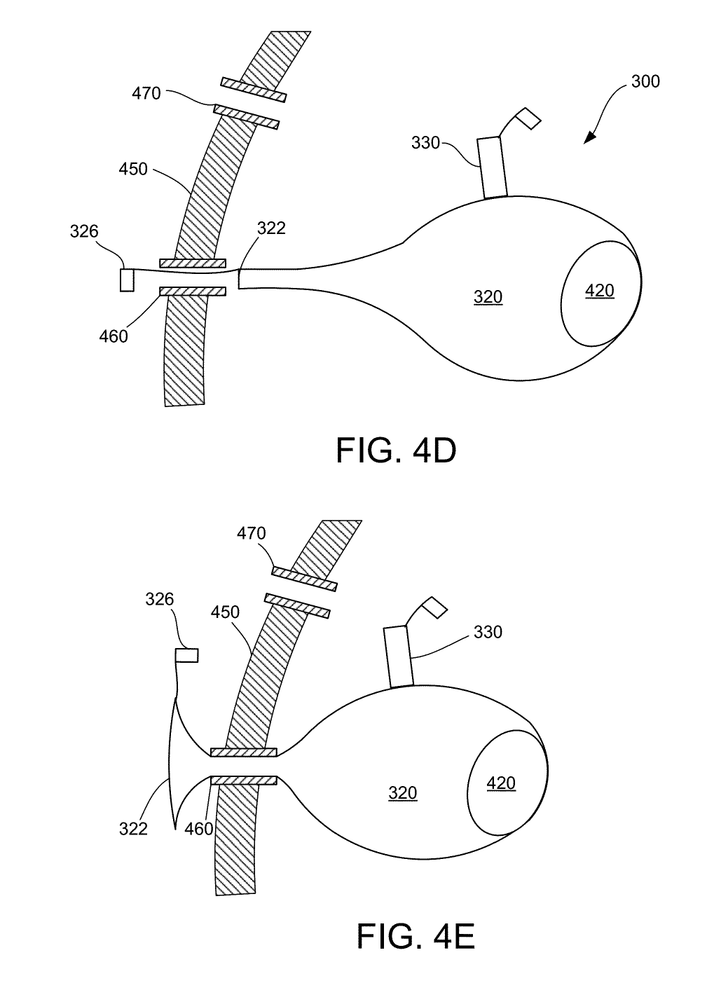

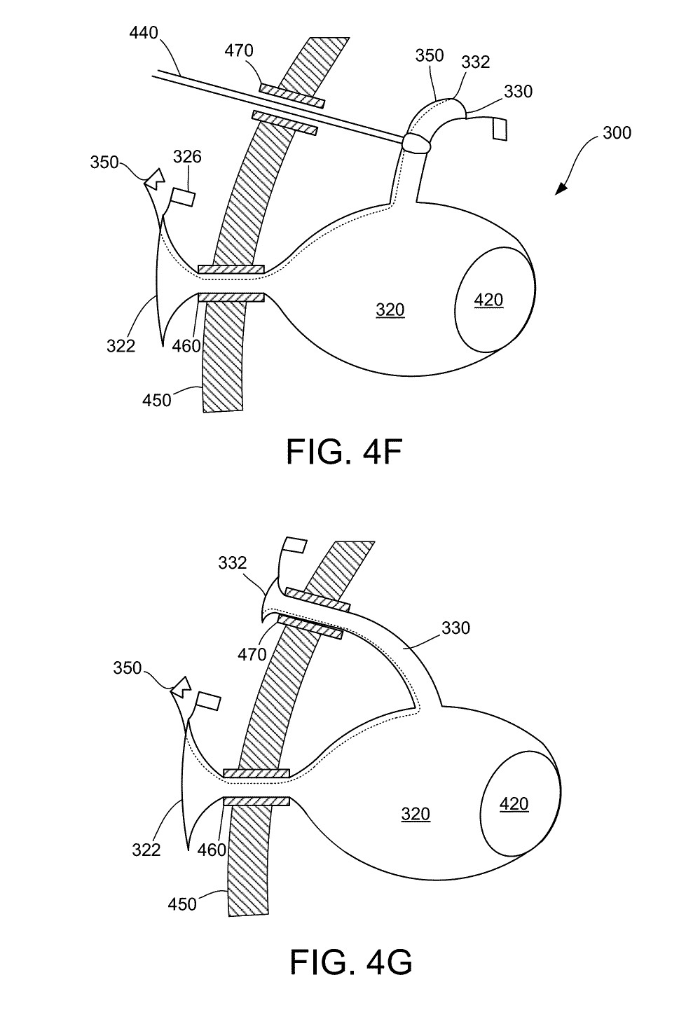

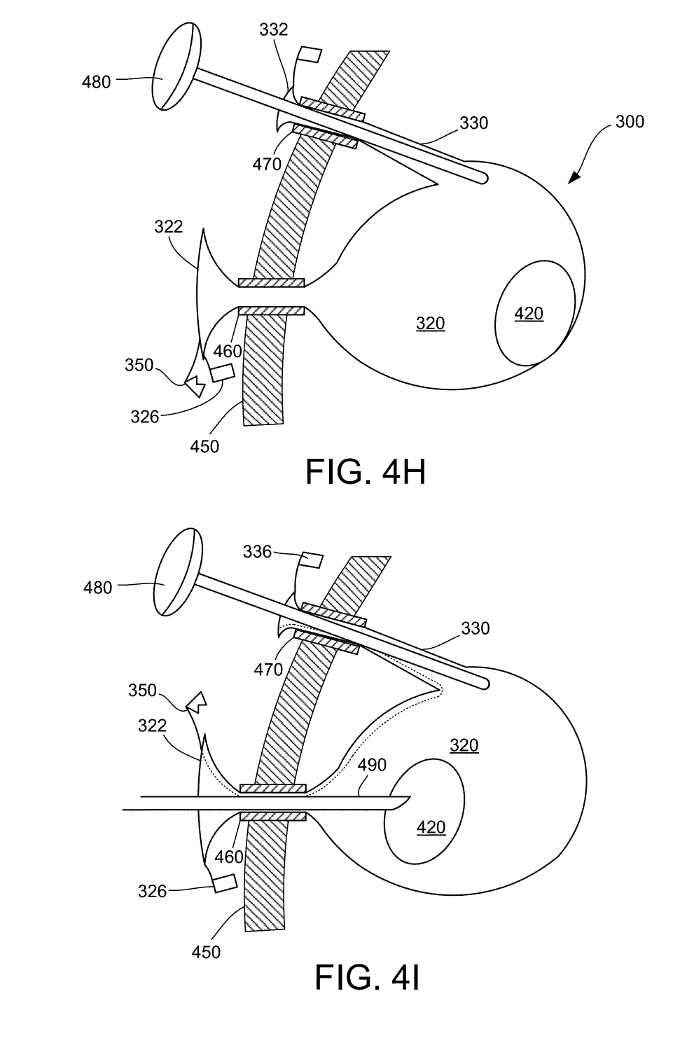

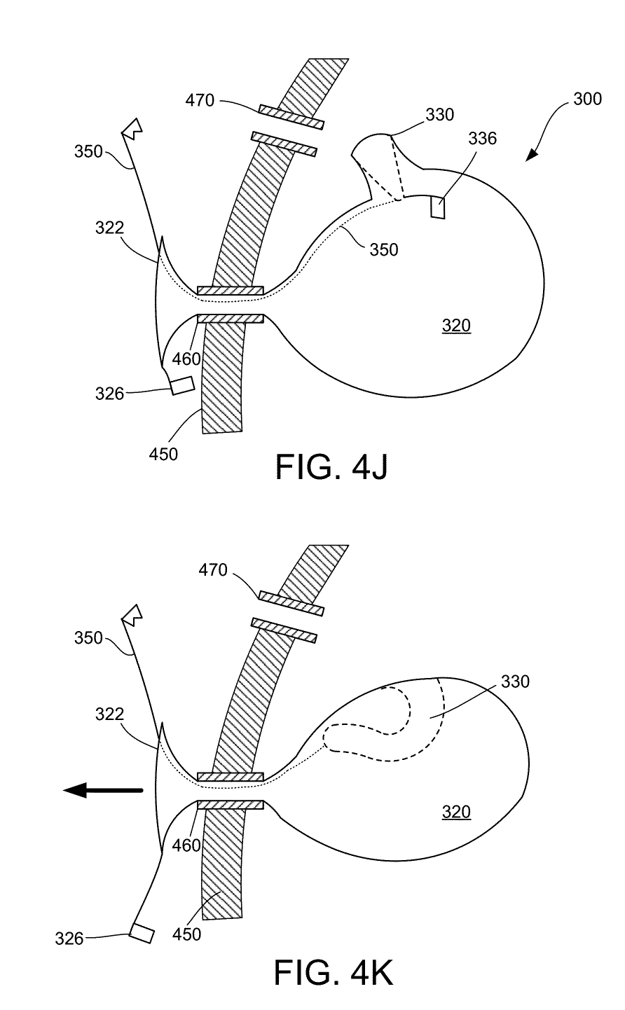

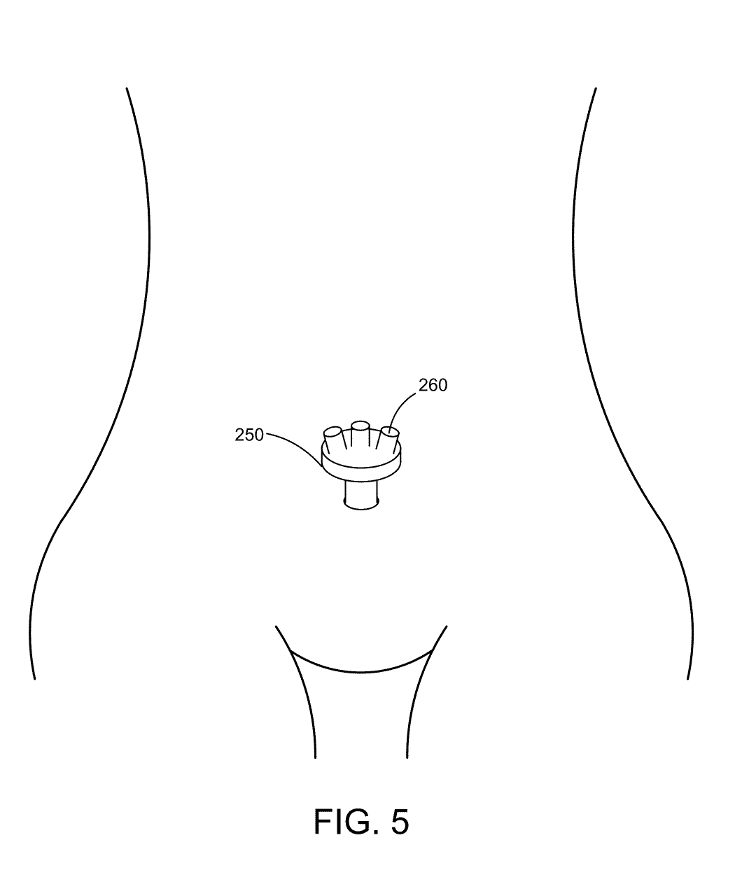

The retraction mechanism 350 may be constructed from a variety of materials that span from the end of the conduit 330, proximate to the secondary opening 332, to the interior of the pouch 320 proximate to the main opening 322, allowing a user to retract the conduit 330 from the main opening 322, as discussed below with respect to FIG. 4J. For example, the retraction mechanism can include a ribbon of fabric, e.g., nylon, that is coupled proximate to the secondary opening 332 and the main opening 322. When the ribbon is pulled from the proximal end, proximate to main opening 322, the conduit 330 will be retracted into the pouch 320. Other materials, such as string, tape, or cord can also be used to couple the secondary opening 332 to the main opening 322, i.e., to be used as a retraction mechanism 350. In some embodiments, the retraction mechanism 350 may be coupled to the closure mechanism 334 such that actuation of the retraction mechanism 350 reduces the size of the secondary opening 332 and retracts the conduit 330 into the pouch 320. A similar retraction mechanism 350 may be included for each additional conduit, e.g., conduit 340. In some embodiments, a unitary retraction mechanism may be included that simultaneously retracts all of the conduits 330, 340, etc. with a single retraction mechanism 350.

An exemplary use of a surgical capture device 300 is shown in FIGS. 4A-4K. The device 300 is initially deployed through a trocar into a surgical cavity, where a tissue 420 will be removed. As explained above, with respect to laparoscopic hysterectomy, several steps will precede delivering the device 300 to the subject, such as evaluating the surgical space and dissecting the tissue 420 to be removed away from other tissues. As shown in FIG. 4A, the initial step is to secure the tissue 420 with a tool, such as laparoscopic forceps 440, so that the tissue 420 can be move through the main opening 322 into the interior of the pouch 320. This step is typically performed in an insufflated cavity while viewing the procedure with a laparoscope 480 (not shown in FIG. 4A). The tissue 420 is typically placed toward the back of the pouch 320, as shown in FIG. 4B. Once the tissue 420 has been placed within the device 300, the main opening 322 can be reduced in size by manipulating the closure mechanism 324. As shown in FIG. 4C, the closure mechanism may include a pull-cord 326 that can be pulled with a tool, e.g., laparoscopic forceps 440, to reduce the size of the main opening 322 thereby securing the tissue 420 within the device. While a contiguous piece of tissue 420 is depicted in FIGS. 4A-4K, it is understood that tissue 420 may include an entire organ, e.g., an uterus or gallbladder, or portions of tissue, e.g., uterine fibroids, cancerous legions, or tumors, or any combination of tissues that are to be removed.

Once the tissue 420 has been secured within the device 300, the main opening 322 of the device 300 is extracted through a first port 460 in the body 450 of the subject undergoing the procedure, as shown in FIG. 4D. Typically the port 460 comprises a trocar configured for laparoscopic access, i.e., as shown in FIG. 2. The port 460 need not be limited to a standard laparoscopic port, however, as any well-defined opening will provide access to the surgical cavity and sufficient space to extract the main opening. In some embodiments, as shown in FIG. 4D, the main opening 322 may be extracted by pulling a pull-cord 326 that brings the entire main opening 322 up and through the first port 460. Once the main opening 322 has been extracted to the exterior of the subject, the main opening 322 can be reopened, as shown in FIG. 4E to provide better access for subsequent steps, described below. In some embodiments, the device 300 includes a self-opening main opening 322 so the main opening will maximize its size automatically after it emerges from the first port 460. In other embodiments, it may be necessary to reopen main opening 322 manually.

Once the main opening 322 has been extracted, the secondary opening 332 of the conduit 330 can be extracted through a second port (or trocar or trocar site) 470, typically a laparoscopic trocar. As shown in FIG. 4F, the conduit 330 can be secured directly with a tool, e.g., laparoscopic forceps 440, or the closure mechanism 334 or a pull cord 336 attached to the closure mechanism 334 can be grabbed and extracted via the second port (or trocar or trocar site) 470. Once the secondary opening 332 is brought through the second port 470, the secondary opening is opened to allow insertion of a tool and/or trocar directly into the interior of the pouch 320, as shown in FIG. 4G. If the device 300, 310 includes more than one conduit 330, the other conduits are also extracted in a similar fashion, providing access to the interior of the pouch for other tools (not shown in FIGS. 4A-4K).

After the secondary opening 332 has been brought to the exterior of the subject, a tool can be inserted through the conduit 330 to access the interior of the pouch 320, as shown in FIG. 4H. As depicted in FIG. 4H, the tool may be a laparoscope 480, allowing a surgeon to visualize the interior of the pouch 320 including the tissue 420 to be removed. The pouch 320 may be insufflated with a gas, e.g., carbon dioxide, as needed to increase the working volume inside the pouch 320. The insufflation gas may be delivered through the single conduit 330 along with the laparoscope 480, of the insufflation gas may be delivered through another port via an additional conduit 350 (not shown in FIG. 4H). Once the tissue 420 has been visualized, a tool 490 to morcellate the tissue 420 can be inserted through the main opening 322 as shown in FIG. 4I. The tool 490 may be a commercial power morcellator, or it may be another tool to minimize the size of the tissue 420 so that it can be removed via the first port 460. For example, the tissue 420 may be cut, cauterized, stripped, ground, or vaporized using another tool. Once the tissue 420 has been removed from the device 300, the tool 490 can be retracted through the first port 460. At this point the laparoscope 480 can be used to visualize the interior of the pouch 320 to assure that all tissue was removed, and to assure the integrity of the pouch 320 has not been compromised in any way.

Once the interior of the pouch 320 has been inspected, the laparoscope 480 can be retracted through the second port 470 and then the secondary opening 332 of the conduit 330 can be closed to assure that nothing escapes the pouch 320 when the device 300 is removed. For example, before the conduit 330 is retracted back into pouch 320, a surgeon may roll the end of the conduit 330 outside the patient as the tool or trocar is being removed, close the secondary opening 332 with the second closure mechanism 334 and tie a knot around the end of the conduit using the tail of the closure mechanism 334t or another piece of suture, etc. (not shown in the FIGS.) In some embodiments, the closure mechanism 334 may be actuated separately from the retraction mechanism 350, as shown in FIG. 4J. In other embodiments, the secondary opening 332 may closed at the same time the conduit 330 is retracted by actuating the retraction mechanism 350. In yet another embodiment (not shown in the FIGS.), the conduit 330 may not be retracted into the pouch before the device is extracted from the subject. In this embodiment, the conduit 330 will be closed off, and the conduit 330 will simply be pulled back through the second port 470 as the device is removed via the first port 460. This process may be facilitated by grasping the distal end of the conduit 330 with a grasper and guiding it back through the secondary port 470. As shown in FIG. 4J, the conduit 330 is inverted and sutured closed before it is pulled into the pouch 320, thereby assuring that any cells or fluids from the tissue 420 are retained within the device 300 as the device is removed from the subject. Once the conduit 330 is fully retracted, the device 300 can be completely removed from the subject by pulling the device through the first port 460 as shown in FIG. 4K. Once removed, the device 300 can be disposed of.

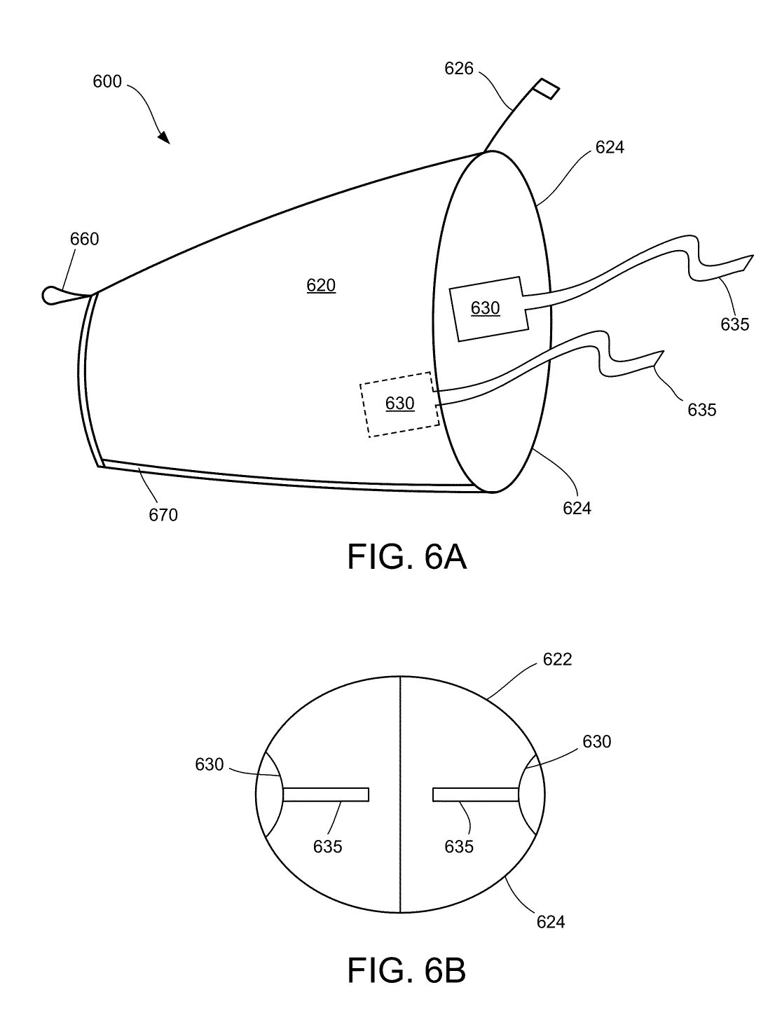

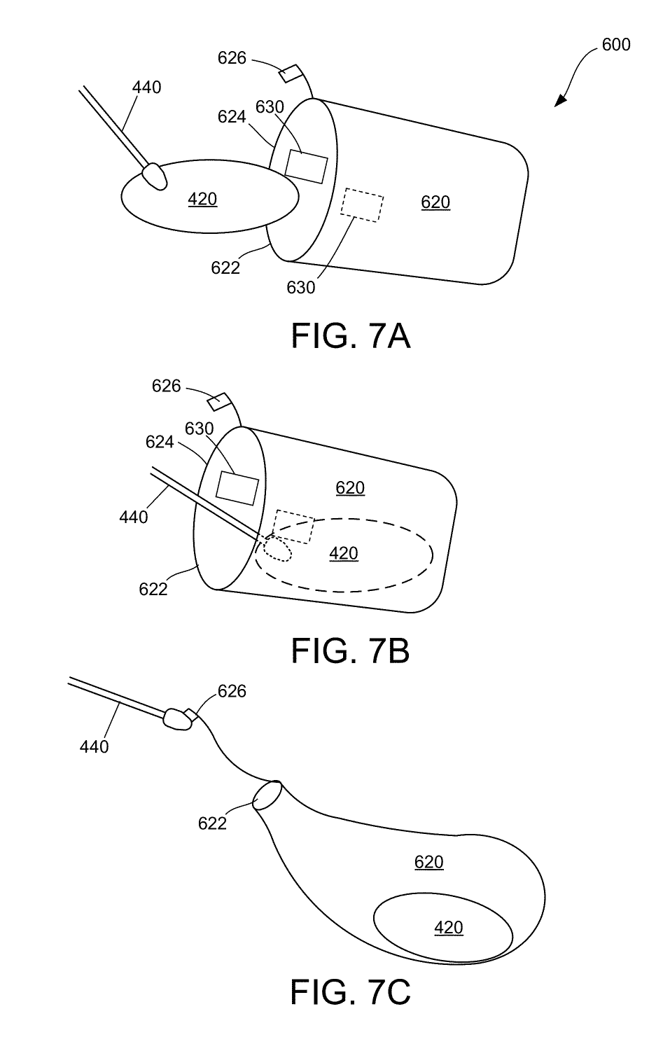

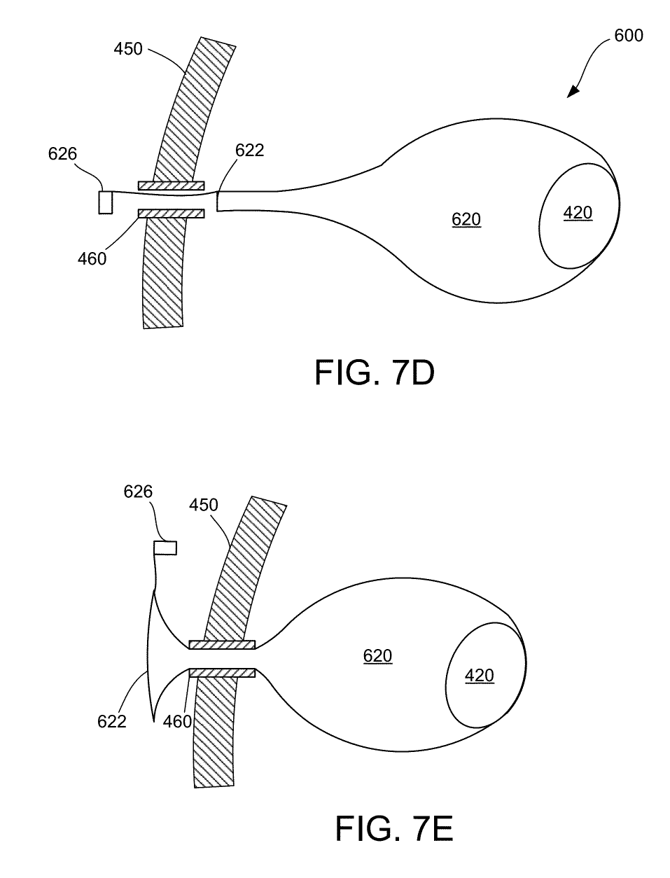

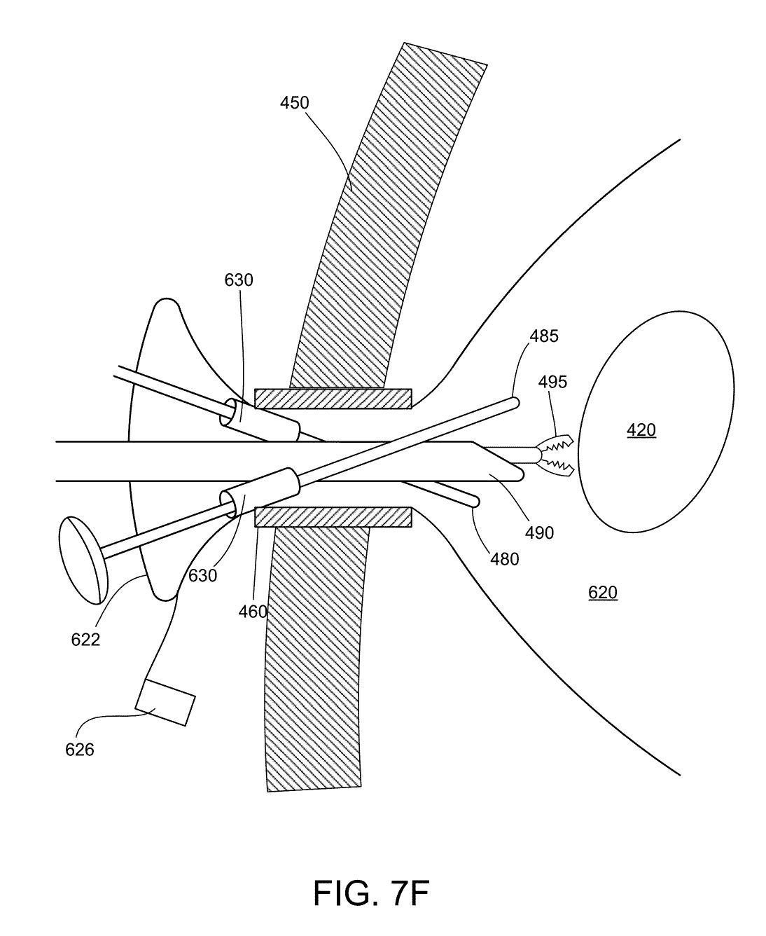

Another embodiment of a surgical capture device consistent with the present disclosure is illustrated in FIGS. 6A-6B and 7A-7F. This embodiment of a surgical capture device is configured to sequester tissue from a surgical cavity while the tissue is morcellated, or otherwise reduced in size. The capture device comprises a pouch with an opening and guides affixed near the opening to facilitate placement of multiple laparoscopic instruments. Once the tissue has been removed from the device, the capture device can be removed from the subject, thereby minimizing the risk that any of the morcellated tissue is distributed within the subject. The device is well-suited for procedures such as laparoscopic hysterectomy using power morcellation to remove the uterus and/or uterine fibroids.

As previously described, a laparoscopic hysterectomy procedure may involve placement of trocars in the umbilicus (belly button) and in the lower left and right quadrants of the pelvis, as shown in FIG. 2. However, in other situations, the procedure may be completed through a single incision, using a single trocar or port, as shown in FIG. 5.

Initially the belly is insufflated with carbon dioxide and the uterus is visualized with a laparoscope and surveyed for excessive adhesions or other defects that contraindicate laparoscopic hysterectomy. Once visualized, the uterus can be manipulated with a uterine manipulator while a cutting instrument (laparoscopic scalpel, cautery, laparoscopic scissors, etc.) and forceps are used to dissect the tissues and coagulate and cut the utero-ovarian ligaments. When done with multi-port laparoscopic hysterectomy, the laparoscope and cutting instrument are typically inserted through different ports. When done with a single-port laparoscopic hysterectomy, the laparoscope and cutting instrument are typically inserted through different cannula of the same port.

Once the ligaments are severed, dissection proceeds, and the uterine arteries and veins are identified. The blood vessels are sealed and severed, and the body of the uterus is cut from the cervix resulting in an intact uterus. While a healthy uterus is typically about the size of an avocado, some postpartum uteruses and diseased uteruses are much larger, e.g., the size of a grapefruit or larger. In some laparoscopic hysterectomy procedures, the uterus and the cervix are cut from the pelvic floor and removed, as shown in FIG. 1B.

FIGS. 6A and 6B show two views of a surgical capture device of the invention. FIG. 6A shows a side perspective view, while FIG. 6B shows a top view, looking directly at the opening of the device. The surgical capture device 600 includes a pouch 620 formed by joining sheet material such as fabric or plastic. The device 600 has an opening 622 that provides a large passageway to the interior of the pouch 620. A closure mechanism 624 surrounds the opening 622 and allows a user to reversibly reduce the size of the opening 622. In some embodiments, the closure mechanism 624 is coupled to a pull-cord 626 that makes it easy to actuate the closure mechanism 624 with a tool, such as laparoscopic forceps. Near the opening 622, at least two guides 630 are provided that assist in placement of laparoscopic instruments through the opening 622. The guides 630 may comprise tabs 635 to assist in locating the guides 630 and to facilitate opening the guides when the device is inserted in a port or cannula. Additionally, in many embodiments, the device 600 includes a pull-tab 660 on the exterior of the pouch 620 opposite the opening 622. The pull-tab 660 provides a point to pull the back of the device to either cause it to move within the surgical cavity, or to help stretch out the device 600, for example, for final extraction from the subject.

In some embodiments, the surgical capture device 600, including the guides 630, is made from fabric, such as nylon or polyester. In some embodiments, the fabric is reinforced with additional threads, such as rip-stop nylon fabric. The reinforcement may run in multiple directions in the fabric to produce, e.g., a cross-hatch or diamond pattern. In some embodiments, the reinforcement may include polymer member or metal wires. The reinforcement prevents instruments from perforating the fabric, and in the event that the fabric is perforated, the reinforcements will prevent the fabric from tearing further. In addition to the structural fabric, the surgical capture device 600 may additionally comprise a lining or a coating to make the device 600 impervious to fluids, e.g., blood or other body fluids. In some embodiments, the lining will comprise polyethylene, polypropylene, or polyurethane. The same materials may alternatively, or in addition, be used to coat the fabric to produce a surgical capture device 600 that is impervious to fluids. In some embodiments, the lining may create a semi-permeable barrier in that small molecules, such as water and some gasses can pass through the barrier while larger molecules, such as proteins, nucleic acids, and lipids cannot pass through the barrier.

In an embodiment, the surgical capture device 600 is constructed from a fabric, e.g., rip-stock nylon, and the seams are joined with thread, e.g., using sewing. In alternative embodiments, the seams may be joined with mechanical fasteners or the seams may be joined with adhesive or other joining material such as fabric tape. The guides 630 are typically smaller segments of fabric attached to the interior of the pouch 620 with thread, e.g., using sewing. The tabs 635 may be integrated with the guides 630, for example, the guide 630 and its tab 635 can be cut from a single piece of fabric. In alternative embodiments, the tab 635 may be separately sewn or bonded to the guide. While the guides 630 are shown of similar size in FIGS. 6A and 6B, the guides 630 may be of different widths and lengths, as needed. Additionally, while only two guides are shown in the figures, there is no practical limit to the number of guides that can be included, e.g., three guides, e.g., four guides, e.g., five guides, e.g., six guides, e.g., seven guides, e.g., eight guides.

The size of the devices 600 of the invention can vary substantially depending upon the needs of the surgeon and the size of the tissue to be removed. For example, the device 600 shown in FIGS. 6A and 6B may comprise at least about 100 cm² of fabric, e.g., at least about 150 cm², e.g., at least about 200 cm², e.g., at least about 250 cm², e.g., at least about 300 cm², e.g., at least about 400 cm², e.g., at least about 500 cm², e.g., at least about 1000 cm² of fabric. The insufflated volume of the pouch 620 may be at least about least 1000 cm³, e.g., at least about 1500 cm³, e.g., at least about 2000 cm³, e.g., at least about 2500 cm³, e.g., at least about 3000 cm³, e.g., at least about 3500 cm³, e.g., at least about 4000 cm³. The guide 630 may be at least about 4 cm in length. The guides 630 may be longer, however, e.g., at least about 5 cm, e.g., at least about 6 cm, e.g., at least about 7 cm, e.g., at least about 8 cm, e.g., at least about 9 cm in length. The guide 630 may be at least about 1 cm in width. The guides 630 may be wider, however, e.g., at least about 2 cm, e.g., at least about 3 cm, e.g., at least about 4 cm, e.g., at least about 5 cm, e.g., at least about 6 cm in width. The tabs 635 may be of a variety of lengths, as needed, and may be color or shape coded for easier identification during the surgical procedure. The tabs may be at least about 8 cm in length. The tabs 635 may be longer, however, e.g., at least about 10 cm, e.g., at least about 12 cm, e.g., at least about 15 cm, e.g., at least about 18 cm, e.g., at least about 20 cm in length.

The opening 622 may be at least 30 cm in circumference, to allow the tissue to be morcellated to be easily placed within the pouch using laparoscopic tools, such as laparoscopic forceps. In alternative embodiments, the opening 622 may be greater than 40 cm in circumference, e.g., greater than 50 cm in circumference, e.g., greater than 60 cm in circumference.

A variety of closure mechanisms 624 can be used to reversibly reduce the size of the opening 622. In some embodiments, the closure mechanism is a pull cord, which comprises a single element such as a nylon, polypropylene, or polyvinylidene fluoride. As the pull cord is pulled, the opening 622 is reduced in circumference, allowing a tissue to be secured within the device 600. In some embodiments, the pull cord comprises multiple elements, such as multiple nylon filaments. In some embodiments, the opening 622 may be reinforced with a polymer layer to improve the stiffness of the opening, as opposed to fabric alone, to facilitate opening the device after it has been delivered to the surgical cavity. Embodiments with multiple filaments have a decreased risk of breakage when the pull-cord is pulled, and the additional resilience provided by the extra elements results in a device 600 that is essentially self-opening. The self-opening feature is especially useful because the device is immediately ready to have the tissue deposited after it has been placed through the trocar. In other words, the surgeon does not have to spend additional time opening the opening 622 before the device 600 can be used. Other closure mechanisms 624 may include pull strings, zippers, snaps, or flange-in-channel closures, i.e., ZIP-LOC™-type closure mechanisms. The closure mechanism may have a handle, tab, or flag to make it easier to interact with the closure mechanism during a procedure. The handle, tab or flag may be color-coded or shape-coded to assist in identifying specific closure mechanisms during a procedure. In some embodiments the closure mechanisms will be elongated, such as an elongated pull-cord to provide easier access to the closure mechanism outside of the patient. For example, in some embodiments, a pull cord may be 30 cm in length beyond the length required for the opening 622 to be completely opened. Other embodiments may include a longer pull cord, e.g., at least 40 cm in length, e.g., at least 50 cm in length.

An exemplary use of a surgical capture device 600 is shown in FIGS. 7A-7F. The device 600 is initially deployed through a trocar into a surgical cavity, where a tissue 420 will be removed. As explained above, with respect to laparoscopic hysterectomy, several steps will precede delivering the device 600 to the subject, such as evaluating the surgical space and dissecting the tissue 420 to be removed away from other tissues. As shown in FIG. 7A, the initial step is to secure the tissue 420 with a tool, such as laparoscopic forceps 440, so that the tissue 420 can be move through the opening 622 into the interior of the pouch 620. This step is typically performed in an insufflated cavity while viewing the procedure with a laparoscope 480 (not shown in FIG. 7A). The tissue 420 is typically placed toward the back of the pouch 620, as shown in FIG. 7B. Once the tissue 420 has been placed within the device 600, the opening 622 can be reduced in size by manipulating the closure mechanism 624. As shown in FIG. 7C, the closure mechanism may include a pull-cord 626 that can be pulled with a tool, e.g., laparoscopic forceps 440, to reduce the size of the opening 622 thereby securing the tissue 420 within the device. While a contiguous piece of tissue 420 is depicted in FIGS. 7A-7F, it is understood that tissue 420 may include an entire organ, e.g., an uterus or gallbladder, or portions of tissue, e.g., uterine fibroids, cancerous legions, or tumors, or any combination of tissues that are to be removed.

Once the tissue 420 has been secured within the device 600, the opening 622 of the device 600 is extracted through a port 460 in the body 450 of the subject undergoing the procedure, as shown in FIG. 7D. Typically the port 460 comprises a trocar configured for laparoscopic access, i.e., as shown in FIG. 2. Alternatively, the port 460 may be a specialty resilient port. The port 460 need not be limited to a standard laparoscopic port, however, as any well-defined opening will provide access to the surgical cavity and sufficient space to extract the opening. In some embodiments, as shown in FIG. 7D, the opening 622 may be extracted by pulling a pull-cord 626 that brings the entire opening 622 up and through the port 460. Once the opening 622 has been extracted to the exterior of the subject, the opening 622 can be reopened, as shown in FIG. 7E to provide better access for subsequent steps, described below. In some embodiments, the device 600 includes a self-opening opening 622 so the opening will maximize its size automatically after it emerges from the port 460. In other embodiments, it may be necessary to reopen opening 622 manually.

Once the opening 622 is properly in place, the instruments needed to remove the tissue 420 can be inserted through the opening 622 using the guides 630 to organize the instruments and facilitate insertion, as shown in FIG. 7F. Typically the first tool to be inserted will be an insufflation tube 485 to fill the pouch 620 with gas, making more working space for the tissue 420 removal. Next a laparoscope 480 will be inserted to allow the surgeon to visualize the interior of the pouch 620 including the tissue 420 to be removed. Both the insufflation tube 485 and the laparoscope 480 will be inserted through guides 630 to keep them organized and clear of the power morcellator 495, as shown in FIG. 7F. Other configurations are also possible, for example, the insufflation gas may be delivered along with the laparoscope 480. Other instruments, such as forceps (not shown) can also be inserted through the opening 622.

Once the tissue 420 has been visualized and the integrity of the pouch 620 has been verified, a power morcellator 495, or other tool, can be used to morcellate the tissue 420 so that it can be removed. For example, the tissue 420 may be cut, cauterized, stripped, ground, or vaporized using another tool. Once the tissue 420 has been removed from the device 600, the power morcellator 495 can be retracted through the port. At this point the laparoscope 480 can be used to visualize the interior of the pouch 620 to assure that all tissue was removed, and to assure the integrity of the pouch 620 has not been compromised in any way.

Once the interior of the pouch 620 has been inspected, the laparoscope 480 can be retracted through the port. Once all of the instruments have been removed, the device 600 can be completely removed from the subject by pulling the device through the port 460. Once removed, the device 600 can be disposed of.

Thus, the various embodiments of the device of the invention provides a surgeon a tool to sequester tissue 420 during a removal process to assure that cells, fluids, etc. are not unintentionally distributed within the subject. The device will reduce procedure times because the surgeon does not have to irrigate and clean up the morcellated tissue fragments from the cavity at the end of the procedure. Additionally, the incidence of inadvertent distribution of malignancies will be virtually zero, provided that the device remains intact during the procedure. Once the device is removed from the subject, the surgical field can be closed as normally done in laparoscopic procedures.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A surgical capture device comprising:
a pouch having a first opening, the first opening providing access to an interior of the pouch;
a first closure mechanism proximal to the first opening and configured to reversibly reduce the size of the first opening; and
a conduit having first and second ends, wherein the first end is coupled to the pouch, and the second end comprises a second opening providing access to the interior of the pouch.

2. The device of claim 1, wherein the pouch comprises nylon fabric.

3. The device of claim 1, wherein the pouch comprises at least 100 cm² of rip-stop nylon.

4. The device of claim 1, wherein the pouch comprises a semi-permeable or water-impermeable material.

5. The device of claim 1, wherein the pouch comprises rip-stop nylon coated with a semi-permeable or water-impermeable material.

6. The device of claim 1, further comprising a second closure mechanism proximal to the second opening and configured to reversibly reduce the size of the second opening.

7. The device of claim 1, wherein the first closure mechanism comprises a pull-cord.

8. The device of claim 7, wherein the pull-cord comprises nylon monofilament.

9. The device of claim 8, wherein the pull-cord comprises two separate nylon monofilaments.

10. The device of claim 7, wherein the pull cord is at least 40 cm in length.

11. The device of claim 1, wherein the first opening is configured to open without assistance after the first opening has been crushed, rolled, or wadded.

12. The device of claim 1, further comprising a retraction mechanism configured to pull the conduit into the interior of the pouch when the retraction mechanism is retracted.

13. The device of claim 12, wherein the retraction mechanism is selected from a cord, a string, and a ribbon.

14. The device of claim 1, wherein the pouch defines a volume of at least 1000 cc³ when the pouch is insufflated.

15. The device of claim 1, wherein the pouch defines a volume of at least 2500 cc³ when the pouch is insufflated.

16. The device of claim 1, further comprising a pull tab coupled to the pouch at an end distal to the first opening.

17. The device of claim 1, further comprising a rigid handle coupled to the first opening.

18. The device of claim 1, wherein the pouch comprises sheet material joined together with seams that are sewn.

19. The device of claim 1, further comprising an additional conduit having third and fourth ends, wherein the third end is coupled to the pouch, and the fourth end comprises a third opening providing access to the interior of the pouch.
